(19) United States
(12) Patent Application Publication
(10) Pub. No.: US 2010/0016607 A1
(43) Pub. Date: Jan. 21, 2010

(54) Process for the Synthesis of Highly Active Binary Metal Fluoride as a Fluorinating Agent for Aromatics

(75) Inventors: William R. Dolbier, JR., Gainesville, FL (US); Krishna Murthy Janmanchi, Santa Barbara, CA (US)

Correspondence Address: SALIWANCHIK LLOYD & SALIWANCHIK;A PROFESSIONAL ASSOCIATION, PO Box 142950, GAINESVILLE, FL, 32614, US

(73) Assignee: University of Florida Research Foundation Inc., Gainesville, FL (US)

(21) Appl. No.: 12/518,751

(22) PCT Filed: Dec. 11, 2007
(86) PCT No.: PCT/US2007/025435

§ 371 Date: Jun. 11, 2009

Related U.S. Application Data
(60) Provisional application No. 60/869,371, filed on Dec. 11, 2006.

(51) Int. Cl. C07D 213/61; C01F 7/50; C07D 213/26; C07C 25/13

(52) U.S. Cl. 546/345; 423/465; 546/346; 570/127; 570/129

(57) ABSTRACT

The subject invention relates to a process for the synthesis of highly active binary metal fluoride system for the fluorination of aromatic compounds. Fluorinated aromatic compounds are valuable synthons for the chemical synthesis of pharmaceutical drugs and novel polymers. Fluorobenzene is used to control carbon content in steel manufacturing, is an intermediate for pharmaceuticals, pesticides and other organic compounds. Fluorobenzene is typically produced by the reaction of aniline and sodium nitrite in the presence of hydrogen fluoride. The present invention relates to a process for the synthesis of highly active binary metal fluoride system consists of copper (II) fluoride and aluminum (III) fluoride for the fluorination of aromatic compounds in gas phase and recycling of the reagent, in situ, using O₂ and HF.

### CROSS-REFERENCE TO RELATED APPLICATION

This application claims the benefit of U.S. Provisional Application Ser. No. 60/869,371, filed Dec. 11, 2006, the disclosure of which is hereby incorporated by reference in its entirety, including all figures, tables and amino acid or nucleic acid sequences.

### BACKGROUND OF THE INVENTION

Hitherto, few methods were reported in patent literature on the fluorination of aromatics using inorganic metal fluorides. U.S. Pat. Nos. 6,087,543 and 6,166,273 teach an improved method for the fluorination of aromatic compounds. However, these patents provide relatively low yields of desired compounds. Patent application publication US 20050096489 deals with physical mixing of commercial metal fluorides as a fluorinating agent. (i.e., CuF₂ with AlF₃, MgF₂, CaF₂). However, relative to the present invention, higher amounts of CuF₂ were employed in the '489 publication and recycling of the material was not studied.

Typically commercial metal fluorides have a tendency to form regular crystallites due to their high lattice energies. Therefore upon thermal treatments the metal fluoride surface areas are in the order of 10-60 m²/g comparable to aluminum fluorides. The fluorination ability of CuF₂ depends on its surface area and prevention of Cu sintering at high temperature reaction conditions. Normally commercial metal fluorides are treated at high temperature and possess thermally stabilized structures. Mechanical mixing of commercial fluorides coupled with high temperature treatment leads to the fluorine loss.

When CuF₂ is in interaction with AlF₃ (as a support) it is possible to obtain high dispersion of CuF₂ on AlF₃ and sintering can be prevented, even at high temperature. Oxidative fluorination of aromatic compounds using transition metal fluorides is schematically represented below.

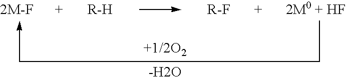

With the simple metal fluorides, the fluorinating power depends on the redox potentials of the metal ions involved. Fluorides of the metal ions with E⁰>1 are very strong fluorinating agents giving rise to saturated products. Fluorides of the metal ions with E⁰<0 are inert towards aromatics. On the other hand, fluorides of the metal ions with 1>E⁰>0 are mild fluorinating agents. Metal fluorides useful as mild fluorinating agents are CuF₂, AgF, HgF₂ and Hg₂F₂.

Fluorination of aromatics using inorganic metal fluorides in gas phase is a “solid-gas” reaction. Normally in solid-gas reactions, once the surface CuF₂ has become reduced to Cu(0), it becomes difficult to diffuse the aromatic compound into the bulk fluorinating reagent due to sintering of copper at high temperature. By selecting aluminum fluoride, an acidic (Bronsted and/or Lewis) support to deposit copper fluoride, it is possible to obtain highly dispersed copper fluoride species on aluminum fluoride.

By applying the Tanabe model (1974) “A New Hypothesis Regarding The Surface Acidity of Binary Metal Oxides” Bull. Chem. Soc. Japan 47:1064-1066) on binary metal fluoride systems, acid generation is caused by an excess of a positive or negative charge in the model structure of a binary fluoride. According to the model, when a divalent metal ion (guest) is doped with a trivalent metal ion (host), Bronsted acidic centers will be formed. Bronsted acid sites will play key role in fluorination reactions (Kemnitz et al. (2002) “Enhanced Lewis acidity by aliovalent cation doping in metal fluorides” Journal of Fluorine Chemistry 114:163-170).

### BRIEF SUMMARY OF THE INVENTION

The subject invention provides a process for the synthesis of highly active copper aluminum fluoride, which is useful in the fluorination of aromatic compounds. Other aspects of the invention provide a method of selecting the precursors for copper and aluminum fluoride. In certain embodiments, inorganic precursors preferably contain nitrates.

Another aspect of the invention provides a method of using a suitable medium, other than water, for the preparation of the binary fluoride. In one embodiment, an organic solvent that dissolves the aluminum nitrate and copper nitrate is used for the preparation of the binary fluoride. One non-limiting example of an organic solvent that can be used is dry ethyl alcohol. Other suitable organic solvents include methanol, ethanol, propanol or butanol.

The subject invention also provides a binary metal fluoride system having better performance in the fluorination of benzene, substituted benzenes (—F, —Cl, —CH₃) and aromatic rings with heteroatoms (N). Also provided is a method wherein the activity and selectivity of the binary fluoride is higher compared to the commercial CuF₂ and CuF₂ supported on AlF₃, which are used under atmospheric operation. The binary fluoride system of the subject invention is expected to have longer life compared to commercial metal fluorides and the reduced metal in the fluorination reaction can be regenerated to metal fluoride using anhydrous HF and O₂.

### BRIEF DESCRIPTION OF THE DRAWINGS

FIG. 1 shows X-ray diffraction (XRD) patterns of CuAl01. Line A depicts the CuAl01 calcined at 500° C; line B represents the CuAl01 reacted with benzene after 17 cycles; and line C shows CuAl01 recycled with HF & O₂ for 4 h after 17 reaction cycles with benzene.

FIG. 2 shows a schematic diagram of reaction/recycle setup.

FIG. 3 depicts the conversion of benzene to fluorobenzene using different metal fluorides (Y-axis: Yield of Fluorobenzene, %).

FIG. 4 illustrates the performance of copper aluminum fluoride in time on stream analysis. 2.5 g of CuAl₂F₈ is reacted with 0.3 ml benzene in each experiment. Ar flow: 30 ml.

FIG. 5 provides X-ray diffraction patterns of copper aluminum fluoride calcined at 500° C. (line A), reacted with benzene after 18 cycles (line B) and recycled with HF & O₂ for 4 h after 20 cycles (line C).

FIGS. 6A-6F depict SEM/EDS patterns of copper aluminum fluoride calcined at 500° C. (FIG. 6A (EDS pattern) and 6B (SEM)), reacted with benzene after 18 cycles (FIG. 6C (EDS pattern) and 6D (SEM)) and recycled with HF & O₂ for 4 h after 20 cycles (FIG. 6E (EDS pattern) and 6F (SEM)).

### DETAILED DISCLOSURE OF THE INVENTION

The present invention relates to a process for the synthesis of highly active binary metal fluoride system for the fluorination of aromatic compounds. Fluorinated aromatic compounds are valuable synthons for the chemical synthesis of pharmaceutical drugs and novel polymers. For example, fluorobenzene is used to control carbon content in steel manufacturing. It is an intermediate for pharmaceuticals, pesticides and other organic compounds is typically produced by the reaction of aniline and sodium nitrite in the presence of hydrogen fluoride. The present invention relates to a process for the synthesis of highly active binary metal fluoride system containing copper (II) fluoride and aluminum (III) fluoride for the fluorination of aromatic compounds in gas phase and recycling of the reagent, in situ, using O₂ and HF.

The synthesis of active copper aluminum fluoride based fluorinating agents can be performed using inorganic precursors of copper and aluminum in a co-precipitation method that uses aqueous hydrofluoric acid. These fluorinating agents provide one with the ability to cause high yield conversion of aromatics to fluorinated aromatics. These fluorinating agents can be recycled using O₂ and anhydrous hydrofluoric acid and can also be used in multiple cycles in the aromatic fluorination processes disclosed herein.

Without being bound by scientific principle, it is believed that the high activity and selectivity of copper aluminum fluoride in the fluorination of aromatic compounds, as described in the present application, is associated with generation of dispersed copper and its interaction with aluminum.

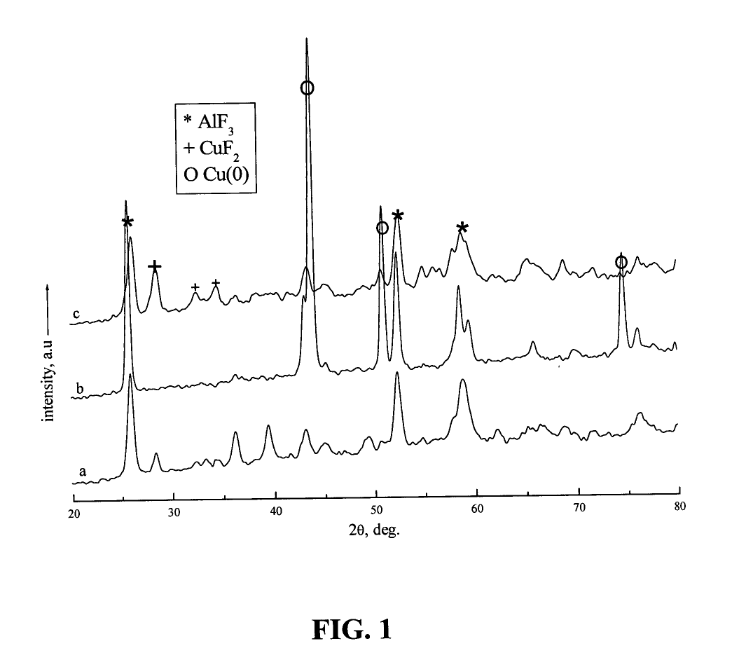

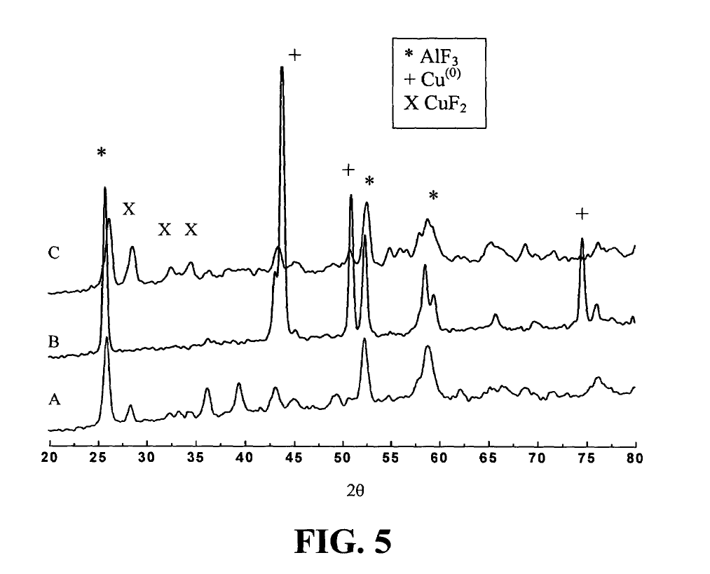

Thus, the subject invention provides the following non-limiting embodiments:
1. A method of making a binary fluoride (CuAl01) comprising:
a) mixing a solution of a copper precursor and an aluminum precursor to form a copper/aluminum admixture and adding said copper/aluminum admixture to a hydrofluoric acid solution;
b) recovering the precipitate formed from step a) and washing said precipitate;
c) drying said precipitate; and
d) calcining said precipitate to form a binary fluoride (CuAl01);
2. The method according to embodiment 1, wherein said copper precursor is copper nitrate, another suitable copper precursor (or combinations of copper precursors) and said aluminum precursor is aluminum nitrate, another suitable aluminum precursor (or combinations of aluminum precursors) and the copper and aluminum precursors can be premixed together prior to the addition of a solvent (e.g., solid aluminum and copper precursors are combined and then mixed with an organic solvent). Alternatively, the aluminum precursor (or combinations thereof) can be dissolved in an organic solvent and the copper precursor (or combinations of copper precursors) can be dissolved in an organic solvent and the two precursor solutions can then be admixed. Other suitable inorganic precursors include aluminum/copper chlorides or aluminum/copper sulfates. A set forth above, mixtures of the various precursors can also be used for form a binary fluoride. The various combinations of precursors are set forth in the following matrix where copper nitrate is designated by “1”; copper chloride is designated by “2”; copper sulfate is designated by “3”; aluminum nitrate is designated by “4”; aluminum chloride is designated by “5”; and aluminum sulfate is designated by “6” (the absence of a precursor being designated by “0”):

| | | | | | | |
|---|---|---|---|---|---|---|
| 1.2.0.0.0.0 | 0.2.3.0.0.0 | 0.0.3.4.0.0 | 0.0.0.4.5.0 | 0.0.0.0.5.6 | 1.0.0.0.5.0 | 0.0.3.0.0.6 |
| 1.0.3.0.0.0 | 0.2.0.4.0.0 | 0.2.0.0.5.0 | 0.2.0.0.0.6 | 1.0.0.4.0.0 | 0.0.3.0.5.0 | 0.0.0.4.0.6 |
| 1.0.0.0.0.6 | 0.2.0.4.0.6 | 0.0.3.4.5.0 | 0.0.3.0.5.6 | 0.0.0.4.5.6 | 0.2.0.0.5.6 | 0.0.3.4.0.6 |
| 1.2.3.0.0.0 | 1.2.0.4.0.0 | 1.2.0.0.5.0 | 1.2.0.0.0.6 | 1.0.3.4.0.0 | 1.0.3.0.5.0 | 1.0.3.0.0.6 |
| 1.0.0.4.5.0 | 1.0.0.4.0.6 | 1.0.0.0.5.6 | 0.2.3.4.0.0 | 0.2.3.0.5.0 | 0.2.3.0.0.6 | 0.2.0.4.5.0 |
| 1.2.3.4.0.0 | 1.0.3.4.5.0 | 0.2.3.0.5.6 | 1.2.3.0.5.0 | 1.0.0.4.5.6 | 0.2.3.4.0.6 | 1.2.3.0.0.6 |
| 1.0.3.0.5.6 | 1.0.3.4.0.6 | 0.2.3.4.5.0 | 1.2.0.4.5.0 | 0.2.0.4.5.6 | 1.2.0.4.0.6 | 0.0.3.4.5.6 |
| 1.2.0.0.5.6 | 1.2.3.4.5.0 | 1.2.3.4.0.6 | 1.2.3.0.5.6 | 1.2.0.4.5.6 | 1.0.3.4.5.6 | 0.2.3.4.5.6 |
| 1.2.3.4.5.6 | | | | | | |

Where a copper or aluminum precursor is absent in the mixture matrix (i.e., “1”, “2” and “3” are “0” or “4”, “5”, and “6” are “0”, selected combinations of these mixtures of copper or aluminum precursors will be mixed together such that at least one aluminum precursor and at least one copper precursor is present in the mixture.
3. The method according to embodiment 1 or 2, wherein said precipitate is dried at about 120° C.;
4. The method according to embodiment 1 or 2 or 3, wherein said precipitate is calcined at about 400° C. to about 500° C.;
5. The method according to embodiment 1 or 2 or 3 or 4, wherein said precipitate is washed with de-ionized water/ethanol;
6. The method according to embodiment 1 or 2, wherein said copper precursor and said aluminum precursor are dissolved in an alcohol or said copper nitrate and aluminum nitrate are dissolved in an organic solvent (and if in separate solutions, the copper precursor and aluminum precursor(s) can be dissolved in the same or a different organic solvent or alcohol);
7. The method according to embodiment 6, wherein said organic solvent is an alcohol (e.g., methanol, ethanol, propanol or butanol);
8. A method of fluorinating an aromatic compound or fluoro/chloroaromatic compound comprising contacting a binary fluoride produced according to the method of embodiments 1-7 with aromatic compound, a fluoroaromatic or chloroaromatic compound, a mixture of aromatic compounds, a mixture of fluoroaromatic and/or chloroaromatic compounds, or a mixture of fluoroaromatic, chloroaromatic and aromatic compounds and heating the contacted compounds to at least 300° C. or at least 350° C. to form a fluorinated product;
9. The method according to embodiment 8, wherein said method further comprises recovering said fluorinated product;
10. The method according to embodiment 8 or 9, wherein said temperature is at least 400° C.;
11. The method according to embodiment 8 or 9, wherein said temperature is at least 425° C.;
12. The method according to embodiment 8 or 9, wherein said temperature is at least 450° C.;
13. The method according to embodiment 8 or 9, wherein said temperature is at least 500° C.;
14. The method according to embodiment 8, 9, 10, 11, 12 or 13, wherein said aromatic or fluoroaromatic compound is selected from the group consisting of benzene, fluorobenzene, chlorobenzene, substituted benzene, substituted fluorobenzene or chlorobenzene, pyridines, fluoropyridines, chloropyridines, substituted pyridines, substituted fluoropyridines or chloropyridines, naphthalene, substituted naphthalenes, fluoronaphthalene, chloronapthalenes, substituted fluoronaphthalenes or chloronapthalenes, toluene, fluorotoluene, chlorotoluene, substituted toluene, and substituted fluorotoluene or chlorotolene;
15. The method according to embodiment 8, 9, 10. 11, 12 or 13, wherein said aromatic compounds are aromatic hydrocarbons;
16. The method according to embodiment 8, 9, 10. 11, 12, 13, 14 or 15, further comprising the recycling of reduced binary fluoride, said recycling comprising contacting the reduced binary fluoride with gaseous HF (or gaseous anhydrous HF) and O₂ at a temperature of at least or about 300° C., at least or about 350° C., at least or about 400° C., at least or about 450° C. or at least or about 500° C.;
17. A binary fluoride produced according to the methods of embodiment 1-7;
18. A binary fluoride having the X-ray diffraction pattern of FIG. 1(a), FIG. 1(b), FIG. 1(c), FIG. 5(a), FIG. 5(b) or FIG. 5(c); or
19. A binary fluoride reagent of the formula CuAl₂F₈.

A method for the conversion of benzene to fluorobenzene is also provided by the subject invention. This method results in at least 50% conversion of benzene with 85% selectivity towards fluorobenzene. The process uses about 2.5 grams of copper aluminum fluoride at atmospheric pressure.

Still another embodiment of the present invention provides a process wherein the recycling of the reduced metal to corresponding metal fluoride, in the presence of HF (e.g., gaseous HF, anhydrous HF, or gaseous anhydrous HF) and O₂, is performed.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### Example 1

### Synthesis of copper Aluminum Fluoride: Activity and Recycling

### Synthesis

A solution of 0.335 mole Cu(NO₃)₂.xH₂O (ALDRICH) in ethanol was added to a solution of 0.665 mole Al(NO₃)₃.9H₂O (ALDRICH) in ethanol. The ethanol solution mixture was then added drop-wise to a 30 ml hydrofluoric acid solution (40% in water) under stirring. After stirring for 1 h, the precipitate was filtered and washed with a small amount of de-ionized water/ethanol, then dried at 120° C. for 12 h and calcined at 400-500° C. for 4-6 h under argon flow (40-60 ml/min). The resultant binary fluoride is designated as CuAl01.

### Activity and Recycling

The fluorination activity of these materials was evaluated in a fixed bed reactor in gas phase. In a typical fluorination experiment, about 2.5 g of catalyst was loaded into a haste alloy reactor tube (8 mm O.D.) in a dry box between two plugs of silver wool. The reactor was placed in an electrically heated furnace and the fluoride material was pre-treated under a flow of argon gas (25-40 ml/min) for 4-6 h at 400-500° C. One-half milliliter to one milliliter of benzene/aromatic compound was introduced into the reactor using a septum in each pass along with the carrier gas (argon, 25-60 ml/min) at 450-500° C. The reactor outlet was connected to a cold-trap and the product mixture coming out of the reactor was condensed in a cold trap kept at 0° C. for 30 minutes for each pass of benzene and the product outlet was connected to an alkali trap to neutralize HF produced as a by-product. The composition of the product mixture was analyzed using gas chromatography equipped with flame ionization detector and H¹NMR & F¹⁹NMR.

After the initial activity experiment (when the conversion of benzene is allowed to fall to zero), the carrier gas was switched off and the reactor is connected to the flow of anhydrous hydrogen fluoride (5-15 ml/min) and oxygen gases (5-20 ml/min) for 2-6 h. Then the reactor was flushed with argon for 0.5-1 h and the reaction cycle (fluorination of aromatics) was repeated without loss of activity during the fluorination step. The flow of the gases was regulated by mass-flow controllers. All the reactor lines were connected using PTFE tubing and heated at 60-80° C.

### Example 2

### Fluorination of Benzene

Fluorination of benzene is carried out utilizing co-precipitated copper aluminum fluoride (CuAl01), commercial CuF₂ (Aldrich), AlF₃ (Aldrich) and a mixture of copper fluoride & aluminum fluoride.

2.5 g of metal fluoride/fluoride mixture was loaded into a haste alloy reactor tube in a dry box. The sample was pre-treated under a flow of argon gas (25 ml/min) for 6 h at 450° C. 0.5 ml of benzene was introduced in to the reactor using a septum in each pass along with the carrier gas (argon, 40 ml/min) at 500° C. The reactor outlet was connected to a cold-trap and the product mixture was condensed for 30 min for each pass of benzene and analyzed using gas chromatography (HP6890) and F¹⁹-NMR. Percentage conversions of benzene to fluorinated products are given in Table 1.

TABLE 1

| | Products and product yields (%) | | | |
|---|---|---|---|---|
| Metal fluoride | 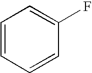 | 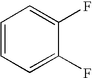 | 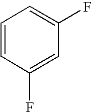 | 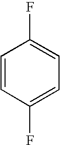 |
| CuF₂ | 11 | 0 | 0 | 0 |
| (Aldrich) | | | | |
| AlF₃ | 0 | 0 | 0 | 0 |
| (Aldrich) | | | | |
| CuF₂ + | 37 | 1 | 3 | 0 |
| AlF₃ | | | | |
| (1:2, | | | | |
| Aldrich) | | | | |
| CuAl01 | 50 | 1 | 5 | 2 |

No conversion to fluorobenzene is observed when benzene is treated with AlF₃ only. Thus, AlF₃ may act as an inert support to disperse CuF₂ and enables larger amount of copper fluoride to react with benzene vapor.

### Example 3

### Recycling of Metal Fluoride

In this example, CuAl01 was reacted with benzene under same conditions as used in Example 1 to form fluorobenzene at 450° C. and 500° C. After the initial activity experiment, the formed copper metal was left in the reactor and treated with anhydrous HF and O₂ for 4 h at 450° C. Then the reactor was flushed with the flow of argon for 30 min and the reaction cycle could be repeated with a single pass of benzene without loss of activity.

TABLE 2

| | % Yield at 450° C. | | % Yield at 500° C. | |
|---|---|---|---|---|
| Experi- ment | 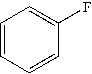 | 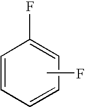 | 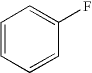 | 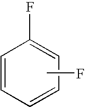 |
| Initial | 25 | 0 | 49 | 8 |
| Recycle | 49 | 0 | 38 | 2 |
| 1 | | | | |
| Recycle | 40 | 0 | 20 | 2 |
| 2 | | | | |
| Recycle | 34 | 0 | 20 | 2 |
| 3 | | | | |

### Example 4

In this example, 1.5 grams of CuAl01 was packed in a haste alloy reactor tube. The binary fluoride was heated to reaction temperature (450° C.) under an argon flow. The argon flow was adjusted to 40 ml/min and 0.5 ml of benzene was introduced in to the reactor using a septum in each pass. The conversion is decreased with each pass due to the formation of copper metal. At the end of the reaction all the CuF₂ was converted to copper metal. The copper metal formed was left in the reactor and treated with anhydrous HF and O₂ for 4 hours at 450° C. The regenerated CuF₂ was again reacted with benzene to form fluorobenzene.

TABLE 3

| | % Yield of fluorobenzene | | |
|---|---|---|---|
| Experiment | Pulse 1 | Pulse 2 | Pulse 3 |
| Initial | 17 | 1 | 0 |
| Recycle 1 | 27 | 10 | 2 |
| Recycle 2 | 25 | 10 | 4 |
| Recycle 3 | 20 | 3 | 1 |

### Example 5

### Time on Stream Studies

2.5 grams of CuAl01 has been continuously tested for the fluorination of benzene at 500° C. with 0.3 ml/pass of benzene at different argon flow rates. After the initial activity experiment, the carrier gas was switched off and an HF/O₂ stream was passed over the reactor at 500° C. for 2.5 h. Then the reactor was flushed with the flow of argon for 30 min and the reaction cycle could be repeated without loss of activity during the fluorination step.

Powder X-ray diffraction analysis (FIG. 1(a)) of fresh CuAl01 (before the fluorination reaction) showed the formation of AlF₃ (major, 2θ=25.32°, 51.96°, 58.13°, ASTM 44-0231) and CuF₂ (weak) signals, which indicates dispersion of Cu²⁺ into AlF₃ lattice. In the inorganic residue, after reaction with benzene, reflections due to metallic copper (2θ=43.33°, 50.47°, 74.20°, ASTM 04-0836) and AlF₃ are observed. It is interesting to observe the crystalline phases due to CuF₂ (2θ=27.59°, 31.66°, 33.68°, ASTM 42-1244) in recycled material, which indicates by oxidizing the reduced metal in the presence of HF, it is possible to regenerate the metal fluoride.

Table 4 shows the product distribution at different recycled experiments.

TABLE 4
CuAl01: 2.5 grams, Reaction temperature: 500° C.,
Benzene: 0.3 ml/pass.

| | | Conversion | Selectivity of | |
|---|---|---|---|---|
| | Flow of | of | fluoro- | Selectivity of |
| | Ar | benzene | benzene | difluorobenzenes |
| Experiment | (ml/min) | (%) | (%) | (%) |
| Initial | 30 | 70 | 65 | 35 |
| Experiment | | | | |
| Recycle 1 | 30 | 46 | 74 | 26 |
| Recycle 2 | 30 | 49 | 75 | 25 |
| Recycle 3 | 30 | 45 | 73 | 27 |
| Recycle 4 | 30 | 48 | 75 | 25 |
| Recycle 5 | 30 | 55 | 77 | 23 |
| Recycle 6 | 30 | 46 | 74 | 26 |
| Recycle 7 | 30 | 48 | 73 | 27 |
| Recycle 8 | 30 | 53 | 71 | 29 |
| Recycle 9 | 30 | 45 | 75 | 25 |
| Recycle 10 | 30 | 38 | 79 | 21 |
| Recycle 11 | 30 | 47 | 74 | 26 |
| Recycle 12 | 42 | 28 | 84 | 16 |
| Recycle 13 | 42 | 19 | 86 | 14 |
| Recycle 14 | 42 | 30 | 85 | 15 |
| Recycle 15 | 42 | 24 | 82 | 18 |
| Recycle 16 | 60 | 14 | 95 | 5 |
| Recycle 17 | 60 | 15 | 95 | 5 |

% Selectivity of ‘x’ = (% Yield of ‘x’/% Total conversion) × 100

### Example 6

### Fluorination of Mono-Fluorobenzene

In this example fluorination of mono-fluorobenzene was carried out using CuF₂, CuF₂+AlF₃ mixture and CuAl01. The experimental conditions as mentioned in Example 1 were followed. The percentage conversions of monofluorobenzene to difluoro derivatives are given in Table 5.

TABLE 5
Weight of the metal fluoride: 2.5 grams, Reaction temperature:
500° C., fluorobenzene: 0.5 ml/pass, argon flow: 40 ml/min

| | Products and product yields (%) | | | |
|---|---|---|---|---|
| Metal fluoride | 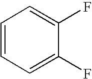 | 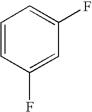 | 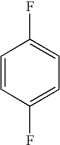 | 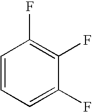 |
| CuF₂ | 1 | 2 | 1 | 0 |
| (Aldrich) | | | | |
| CuF₂ + | 7 | 14 | 9 | 0 |
| AlF₃ | | | | |
| (1:2, | | | | |
| Aldrich) | | | | |
| CuAl01 | 8 | 34 | 17 | 6 |

### Example 7

### Fluorination of Pyridine

In this example fluorination of pyridine was carried out using CuF₂, CuF₂+AlF₃ mixture and CuAl01. The experimental conditions as mentioned in Example 1 were followed. The percentage conversions of pyridine to fluoropyridine derivatives are given in Table 6.

TABLE 6
Weight of the metal fluoride: 2.5 grams, Reaction temperature:
500° C., pyridine: 0.5 ml/pass, argon flow: 40 ml/min

| | Products and product yields (%) | |
|---|---|---|
| Metal fluoride | 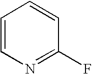 | 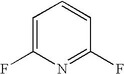 |
| CuF₂ (Aldrich) | 8 | 0 |
| CuF₂ + AlF₃ | 9 | 3 |
| (1:2, Aldrich) | | |
| CuAl01 | 32 | 11 |

### Example 8

### Fluorination of Toluene

In this example fluorination of toluene was carried out using CuF₂, CuF₂+AlF₃ mixture and CuAl01. The experimental conditions as mentioned in Example 1 were followed. The percentage conversions of toluene to fluorotoluene derivatives are given in Table 7.

TABLE 7
Weight of the metal fluoride: 2.5 grams,
Reaction temperature: 500° C., toluene:
0.5 ml/pass, argon flow: 40 ml/min

| | % Yield of |
|---|---|
| Metal fluoride | 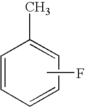 |
| CuF₂ (Aldrich) | 0 |
| CuF₂ + AlF₃ | 4 |
| (1:2, Aldrich) | |
| CuAl01 | 10 |

### Example 9

### Additional Examples

Preparation of the binary reagent was carried out as follows: Four solids of different composition were prepared by using CuF₂:AlF₃ molar ratios of 1:1, 1:1.5, 1:2 and 1:2.5. Appropriate amounts of Al(NO₃)₃.9H₂O and Cu(NO₃)₂.xH₂O were dissolved in ethanol. The mixture was then added drop-wise to a stirred 48% hydrofluoric acid solution. After stirring for 2 h, the precipitate was filtered and washed with a small amount of distilled water/ethanol, then dried at 120 ° C. over night (16 h) and calcined at 450° C. for 6 h under Ar flow. Commercial CuF₂ alone and a physical mixture of CuF₂ and AlF₃ with a ratio of 1:2 were also tested for fluorination of aromatics under identical conditions.

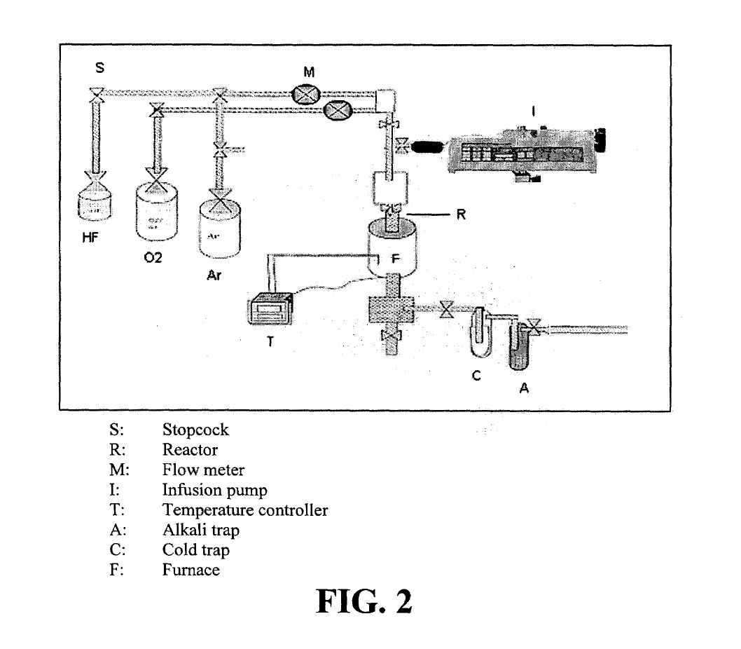

The fluorination activities of these materials were evaluated in a fixed bed reactor in the gas phase (FIG. 2). In a typical fluorination experiment, about 2.5 g of reagent was loaded into a hastalloy reactor tube (8 mm O.D.) in a dry box between two plugs of silver wool. The reactor was placed in an electrically heated furnace and the reagent was pre-treated under a flow of argon gas (40 mL/min) for 4 h at 500° C. Benzene was introduced in to the reactor using a syringe pump (flow rate: 15 mL/min, volume: 0.5 mL) along with the carrier gas (Ar, 25 mL/min) at 450-500° C. The product mixture coming out of the reactor was condensed in a cold trap kept at 0 ° C. for 30 minutes for each pass of benzene and the outlet from this trap was connected to an alkali trap to neutralize the biproduct HF. The composition of the product mixture was determined by gas chromatography and ¹H and ¹⁹F NMR. Fluorobenzene was the only product at 450° C., whereas small amounts of other products such as 1,3- and 1,2-difluorobenzene were also obtained at 500° C.

Once the reagent was expended (i.e, conversion of benzene fell to zero), the carrier gas was switched off and the reactor was connected to a flow of gaseous anhydrous hydrogen fluoride (8 mL/min) and oxygen (10 mL/min) for 2.5 h. Then the reactor was flushed with argon for 0.5 h and the reaction cycle (fluorination of benzene) was repeated in the fluorination process.

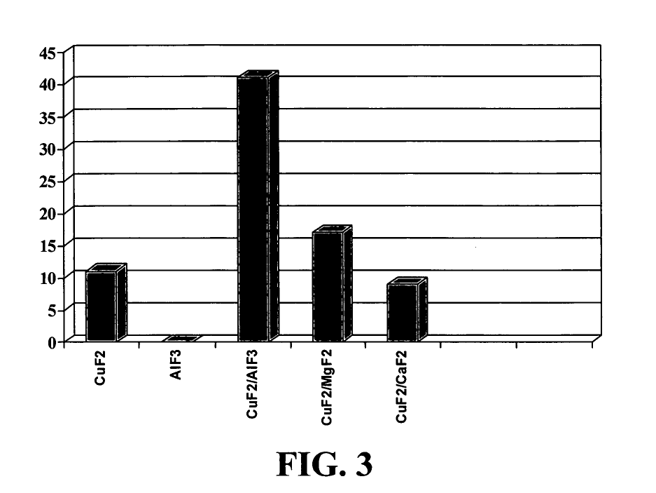

Using this same process, a series of supported and unsupported CuF₂ systems were evaluated with respect to the fluorination of benzene. Pure copper (II) fluoride gave only low conversion (11%) to fluorobenzene, whereas among the supported, physically mixed systems, CuF₂/AlF₃ was the most effective towards fluorination as compared to MgF₂, CaF₂ and AgF giving rise to 41% conversion at 500° C. (FIG. 3). All such metal fluorides were commercial samples (Aldrich). Among the co-precipitated binary fluoride systems, CuF₂:AlF₃ with a ratio of 1:2 was found to exhibit optimal fluorinating ability in the fluorination of benzene (Table 8).

TABLE 8
Conversion of benzene [%]

| CuF₂:AlF₃ ratio | | |
|---|---|---|
| 2.5 g, 500° C. | Initial | Recycled |
| 0.5 ml benzene | activity | activity |
| 1:1 | 28 | 9 |
| 1:1.5 | 53 | 14 |
| 1:2 | 57 | 42 |
| 1:2.5 | 36 | 30 |

Regarding benzene fluorination, AlF₃ was found to be totally inactive, which indicates that it acts only as a support to disperse CuF₂. Comparing the co-precipitated binary reagent CuAl₂F₈ with the CuF₂/AlF₃ mixture, the binary fluoride exhibits greater reactivity (56%) compared to the physically-mixed, commercial CuF₂ and AlF₃ under identical conditions (Table 9).

Fluorination of aromatics using inorganic metal fluorides in gas phase is a “solid-gas” reaction. Normally in solid-gas reactions, once the surface CuF₂ has become reduced to Cu(0), sintering of copper can begin to occur at the high temperature of reaction. As a result of the process of co-precipitating CuF₂ with AlF₃ followed by heating and treatment with HF, it has been possible to obtain a high dispersion of CuF₂ within the AlF₃ support, such that sintering can be minimized, if not prevented, even at high temperature. By selecting aluminum fluoride, an acidic (Lewis acid) support to deposit copper fluoride, it has been possible to obtain highly dispersed copper fluoride species on aluminum fluoride.

The copper aluminum fluoride systems (both the commercial and co-precipitated) were tested in multiple cycles, in the oxyfluorination of benzene. After the initial activity experiment (single pass benzene), the carrier gas was switched off and the reactor is connected to the flow of anhydrous hydrogen fluoride (8 ml/min) and oxygen gases (10 ml/min) for 2.5 h. Then the reactor was flushed with argon for 0.5 h and the reaction cycle (fluorination of benzene) was repeated. The co-precipitated copper aluminum fluoride remained very active and stable during multiple cycles, whereas when using the commercial metal fluoride mixture, the benzene conversion fell to zero within 3 cycles.

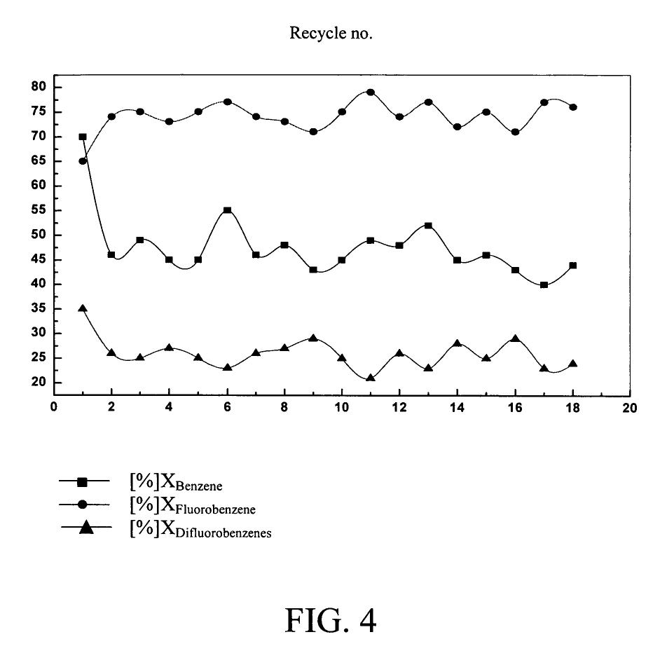

Thus the benzene-CuF₂ (copper aluminum binary fluoride) reaction has been demonstrated to be a two phase, cyclic process in which generation of the CuF₂ in situ from copper metal by successive reaction with oxygen and hydrogen fluoride at 500° C. was followed by reaction with benzene, the cycle being completed by re-conversion of the copper metal residue back to CuF₂ followed by further reaction with benzene to obtain virtually identical yields of fluorobenzenes in each cycle. The binary fluoride system was found to be very stable through 20 recycled experiments, exhibiting constant activity of ˜45% conversion with almost 80% selectivity towards monofluorobenzene (FIG. 4).

X-ray diffraction patterns of copper aluminum binary fluoride are shown in FIG. 5. In the fresh sample (calcined at 450° C. under Ar for 4 h), before reacting with benzene, crystalline phases due to AlF₃ are mainly seen along with very weak signals due to CuF₂ (line A), which indicates that the CuF₂ is mostly dispersed within the AlF₃. In the XRD pattern of the spent system (after reacting with benzene), strong crystalline signals due to Cu(0) appear as a result of the reaction of CuF₂ with benzene to form fluorobenzene (line B). Crystalline signals of CuF₂ and loss of Cu(0) signals in the XRD pattern of the recycled material can be seen. This indicates that CuF₂ has been regenerated by treatment with oxygen and HF.

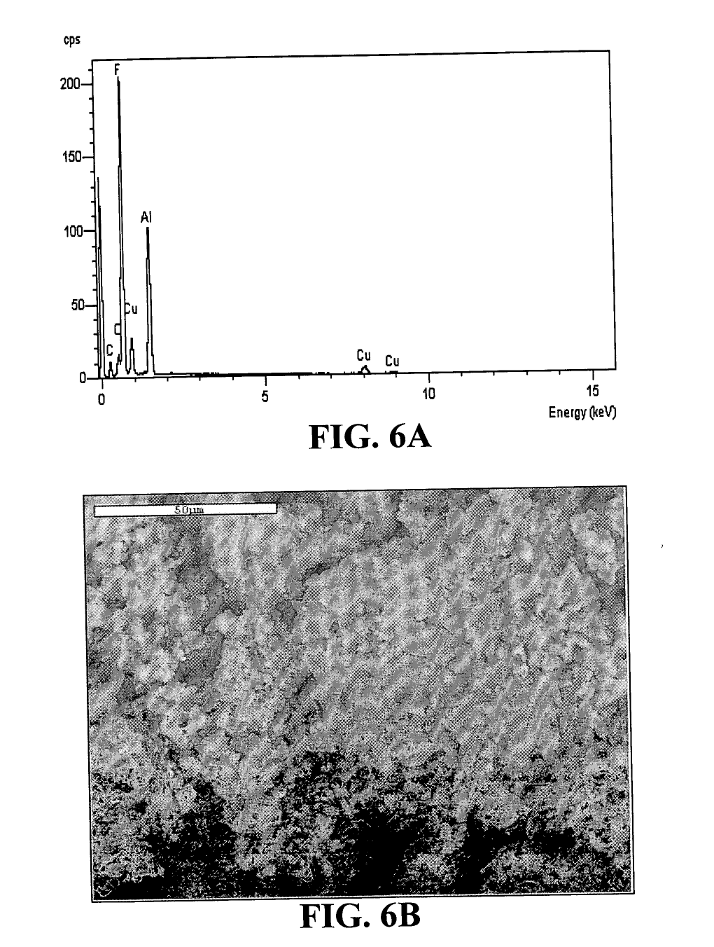

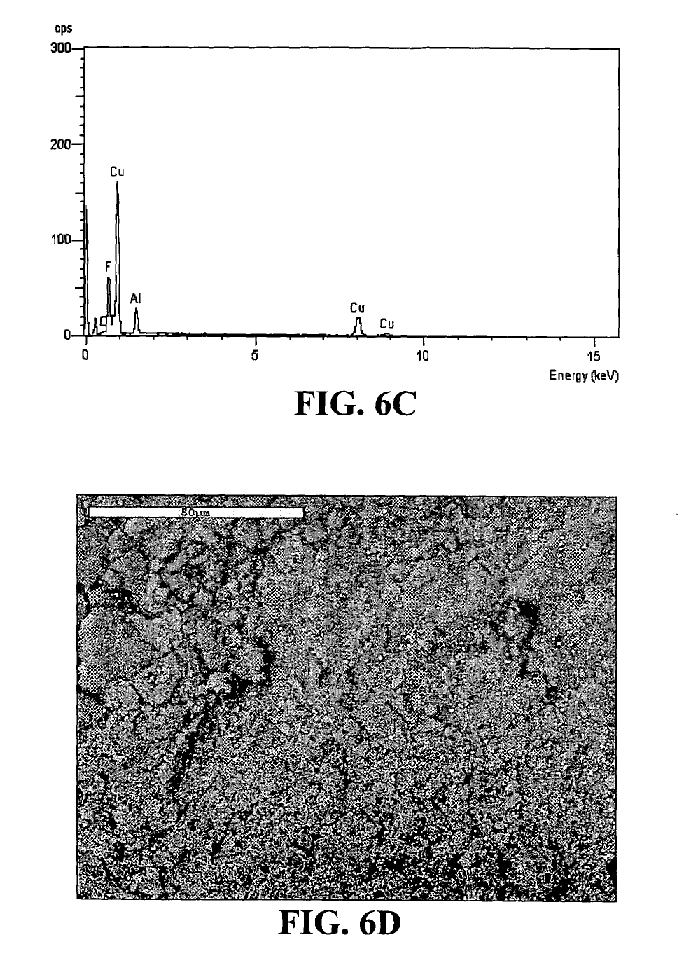

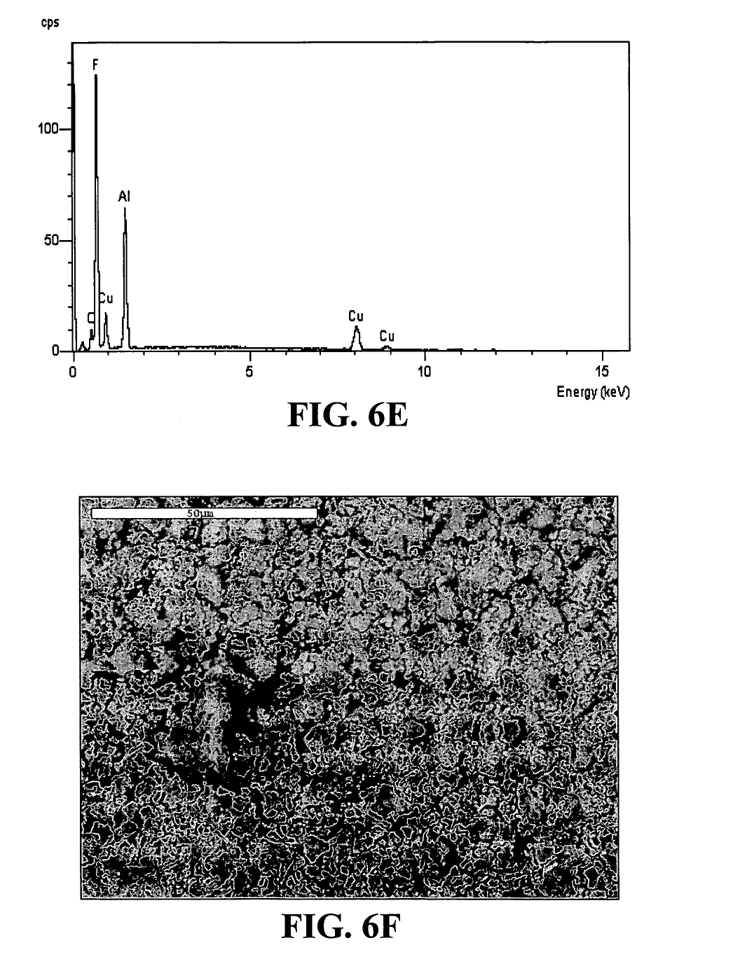

The copper aluminum binary fluoride has also been characterized by scanning electron microscopy (SEM) and energy dispersive spectrometer (EDS). Fresh sample (before reaction with benzene) exhibits uniform morphology and the presence of a high concentration of fluoride on the surface (FIG. 6B). In the partially spent binary fluoride material (after limited reaction with benzene), a fine distribution of copper can be seen (FIG. 6D). This indicates that the copper is not sintered or agglomerated even at the high reaction temperature. Thus, as mentioned above, it has apparently been possible to prevent copper sintering by use of the highly dispersed binary reagent when using limited amounts of benzene, under which conditions the Cu(II) is not exhaustively reduced. In the regenerated binary fluoride material, after 20 cycles, surface analysis shows a uniform morphology with a high concentration of fluoride (FIG. 6F).

This new copper aluminum fluoride reagent has also been found to be active in fluorinating monofluorobenzene and pyridine (Table 9), with 1,3-difluorobenzene and 2-fluoropyridine being obtained as major products in those reactions, respectively. The CuAl₂F₈ reagent has also been found to be effective in converting chloroaromatics to fluoroaromatics. Results for the reactions of 1-chloronaphthalene, chlorobenzene, dichlorobenzenes, chlorotoluenes and chlorofluorobenzenes are summarized below and in Tables 10 and 11.

### Fluorination of 1-chloronaphthalene

### Catalyst: CuAl₂F₈, wt: 2.5 g, reactant: 0.5 ml at 450° C.

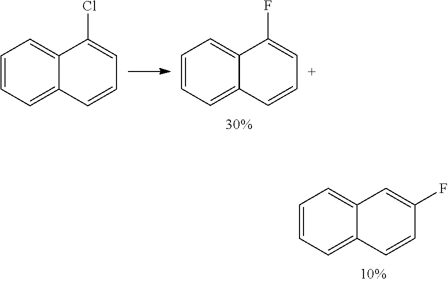

TABLE 9
% Yields of products

| | Reaction with | | Reaction with | | | | |
|---|---|---|---|---|---|---|---|
| | Benzene | | monofluorobenzene | | | Reaction with Pyridine | |
| Catalyst 2.5 g, 500° C. 0.5 ml aromatic compound | 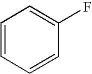 | 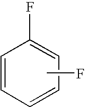 | 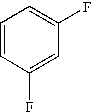 | 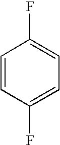 | 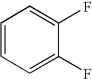 | 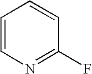 | 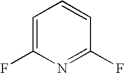 |
| CuF₂ | 11 | 0 | 7 | 3 | 3 | 8 | 0 |
| CuF₂ + AlF₃ | 40 | 1 | 14 | 9 | 7 | 9 | 3 |
| Cu Al₂F₈ | 47 | 10 | 34 | 17 | 8 | 32 | 11 |

TABLE 10
% Yield of Products

| Reactant | | | |
|---|---|---|---|
| 0.5 ml reactant, | | | |
| 2.5 g Catalyst, at | o-fluorotoluene | m-fluorotoluene | p-fluorotoluene |
| o-chlorotoluene | 60 | 7 | 4 |
| m-chlorotoluene | 0 | 61 | 2 |
| p-chlorotoluene | 5 | 10 | 71 |

TABLE 11

| | % Yield of Products | | | | | | |
|---|---|---|---|---|---|---|---|
| Reactants 2.5 g, 500° C. 0.5 ml chloroaromatic compound | 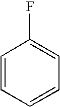 | 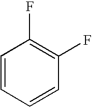 | 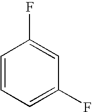 | 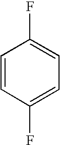 | 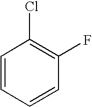 | 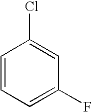 | 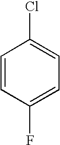 |
| Chlorobenzene | 57 | 1 | 4 | 0 | 3.5 | 2.5 | 0 |
| 1,2- Dichlorobenzene | 10 | 4 | 3 | 1 | 20 | 0 | 0 |
| 1,3- Dichlorobenzene | 6 | 1 | 12 | 0 | 0 | 12 | 0 |
| 1-Chloro-2- Fluorobenzene | 39 | 16 | 21 | 7 | 17 | 0 | 0 |
| 1-Chloro-3- Fluorobenzene | 12 | 0 | 62 | 3 | 0 | 23 | 0 |
| 1-Chloro-4- Fluorobenzene | 4 | 0 | 11 | 42 | 0 | 0 | 43 |

As indicated herein, the newly synthesized copper aluminum fluoride, CuAl₂F₈, exhibits improved activity towards fluorination of aromatic compounds in comparison to the physical mixture of commercial CuF₂ and AlF₃. It has also been shown to maintain reactivity through 20 reaction cycles of regeneration. The high fluorination ability and recyclability of the binary fluoride might be due to the high dispersion of CuF₂ on AlF₃, which combined with the use of limited amounts of benzene in each run seems to inhibit sintering of copper.

There are also certain advantages of the disclosed process. These include: 1) compelling economics; the estimated cost of manufacture is less than half that of the diazonium process; 2) safety; the process design avoids the possibility of runaway reactions that can be encountered when using the diazonium ion based synthesis; and 3) minimal waste; with virtually no waste, the new process is extremely environmentally friendly.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

1-20. (canceled)

21. A method of making a binary fluoride (CuAl01) comprising:
a) mixing a copper precursor and an aluminum precursor to form a copper/aluminum admixture and adding said copper/aluminum admixture to a hydrofluoric acid solution;
b) recovering the precipitate formed from step a) and washing said precipitate;
c) drying said precipitate; and
d) calcining said precipitate to form a binary fluoride (CuAl01).

22. The method according to claim 21, wherein said copper precursor is copper nitrate, copper sulfate, copper chloride or mixtures thereof and said aluminum precursor is aluminum nitrate, aluminum chloride, aluminum sulfate or mixtures thereof.

23. The method according to claim 21, wherein said precipitate is dried at about 120° C.

24. The method according to claim 21, wherein said precipitate is calcined at about 400° C. to about 500° C.

25. The method according to claim 21, wherein said precipitate is washed with de-ionized water/ethanol.

26. The method according to claim 21, wherein:
a) said copper precursor and said aluminum precursor are dissolved together in an organic solvent to form the copper/aluminum admixture; or
b) said copper precursor is dissolved in an organic solvent and said aluminum precursor an organic solvent and then mixed together to form the copper/aluminum admixture.

27. The method according to claim 26, wherein said organic solvent is an alcohol selected from methanol, ethanol, propanol, or butanol and the organic solvent used to dissolve the aluminum precursor(s) and the organic solvent used to dissolve the copper precursor(s) is the same or different.

28. A method of fluorinating an aromatic compound, a chloroaromatic compound or fluoroaromatic compound comprising contacting a binary fluoride produced according to the method of claim 1 with aromatic compound, a fluoroaromatic compound, a chloroaromatic compound, a mixture of aromatic compounds, a mixture of fluoroaromatic and/or chloroaromatic compounds, or a mixture of fluoroaromatic, chloroaromatic compounds and aromatic compounds and heating the contacted compounds to at least 300° C. to form a fluorinated product.

29. The method according to claim 28, wherein said method further comprises recovering said fluorinated product.

30. The method according to claim 28, wherein said temperature is at least 400° C.

31. The method according to claim 28, wherein said temperature is at least 425° C.

32. The method according to claim 28, wherein said temperature is at least 450° C.

33. The method according to claim 28, wherein said temperature is at least 500° C.

34. The method according to claim 28, wherein said aromatic or fluoroaromatic compound is selected from the group consisting of benzene, fluorobenzene, chlorobenzene, substituted benzene, substituted fluorobenzene, substituted chlorobenzene, pyridines, fluoropyridines, chloropyridines, substituted pyridines, substituted fluoropyridines, substituted chloropyridines, naphthalene, substituted naphthalenes, chloronapthalenes, fluoronaphthalene, substituted fluoronaphthalenes, substituted chloronapthalenes, toluene, fluorotoluene, chlorotoluene, substituted toluene, substituted chlorotoluene, substituted fluorotoluene and combinations thereof.

35. The method according to claim 28, wherein said aromatic compounds are aromatic hydrocarbons.

36. The method according to claim 28, further comprising recycling reduced binary fluoride, said recycling comprising contacting the reduced binary fluoride with gaseous anhydrous HF and O₂ at a temperature of about 300° C.

37. The method according to claim 36, further comprising repeating the method of claim 28.

38. A binary fluoride produced according to the method of claim 21.

39. A binary fluoride having the X-ray diffraction pattern of FIG. 1(a), FIG. 1(b) FIG. 1(c), FIG. 5(a), FIG. 5(b) or FIG. 5(c).

40. A binary fluoride of the formula CuAl₂F₈.
